(19) United States
(12) Patent Application Publication
(10) Pub. No.: US 2007/0191694 A1
(43) Pub. Date: Aug. 16, 2007

(54) Methods for measuring moisture content of skin

(76) Inventors: Faiz Feisal Sherman, West Chester, OH (US); Vladimir Gartstein, Cincinnati, OH (US); David Burton Moore, Hamilton, OH (US); Carl Hinz Margraf III, Frankfurt am Main (DE); Brian Keith Fisher, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/514,643

(22) Filed: Sep. 1, 2006

Related U.S. Application Data
(60) Provisional application No. 60/713,813, filed on Sep. 2, 2005.

(51) Int. Cl. A61B 5/00

(52) U.S. Cl. 600/306; 604/290

(57) ABSTRACT

Methods provide for measuring moisture content of skin, such as the scalp. More particularly, the present invention is directed to a method comprising the steps of: (a) providing a means for measuring skin moisture content; (b) using said means for measuring skin moisture content to obtain at least two measured moisture content values from skin; (c) comparing said at least two measured moisture content values with each other to obtain a measured moisture content differential; and (d) correlating said measured moisture content differential to a skin moisture value for said skin.

### CROSS REFERENCE TO RELATED APPLICATIONS

This application claims the benefit of US Provisional application Ser. No. 60/713,813, filed on Sep. 9, 2005.

### FIELD OF THE INVENTION

The present invention relates generally to methods for measuring moisture content of skin. The present invention also relates to methods for measuring moisture content of scalp and methods of treating the scalp. Another aspect of the present invention, relates to methods for measuring dandruff conditions on the scalp of a consumer.

### BACKGROUND OF THE INVENTION

Skin is subject to insults by many extrinsic and intrinsic factors. Extrinsic factors include ultraviolet radiation (e.g., from sun exposure), environmental pollution, wind, heat or infrared radiation (IR), low humidity, harsh surfactants, abrasives, and the like. Intrinsic factors include chronological aging and other biochemical changes from within the skin. Whether extrinsic or intrinsic, these factors result in visible signs of skin aging and environmental damage, such as wrinkling and other forms of roughness (including increased pore size, flaking and skin lines), and other histological changes associated with skin aging or damage. Additionally, the water content of the stratum corneum has a profound influence on the appearance, flexibility, texture, and dryness of the skin, and also on the absorption of drugs and other molecules into and through the skin. The stratum corneum is the outermost layer of the epidermis, and comprises the surface of the skin.

Methods of treating the skin generally involve the application of at least one of a variety of appropriate treatments. Such treatments may be selected to provide or to restore certain desired physical or cosmetic characteristics to the skin or scalp. However, unless an appropriate treatment is selected, the desired physical or cosmetic characteristic may not be obtained.

In the case of treating skin, such as the scalp, treatments generally include shampoos, conditioners, colorants, styling compositions, and the like. Manufacturers of these scalp treatments may provide multiple versions of a type or brand of scalp treatment, wherein each of the multiple versions is specifically designed to target a need or demand which is characteristic of a specific consumer segment and which may be based on physical or cosmetic differences of the scalp generally found between respective consumer segments. For example, a single brand of shampoo may offer a first version designed to treat flakes and a second version designed to treat dryness, both conditions associated with dandruff.

However, when a consumer is faced with the task of selecting a scalp treatment from among the multiple versions of a scalp care brand, the consumer may unknowingly select a version which is not designed to provide the characteristics desired by the consumer. In such a case, the consumer may be dissatisfied with the results of the selected version of the scalp care brand. As a result of the consumer's dissatisfaction, the consumer subsequently may refuse to select any of the versions of that same scalp care brand even though another version of that scalp care brand may provide the consumer's desired scalp and/or hair characteristics. The occurrence of such circumstances, in turn, may lead to unnecessary loss of sales of the particular scalp care brand for the manufacturer.

Methods of measuring moisture content have been developed in the past to determine the moisture level of skin or hair, and have relied on various techniques including resistance and capacitance measurements to obtain the desired indication.

### SUMMARY OF THE INVENTION

It has now been discovered that an accurate and repeatable assessment of hydration characteristics of the skin, including the scalp, is achieved by comparing the value of a parameter, such as skin moisture content, at a first position on the skin, such as a control area that is in equilibrium with environment, with the value of the parameter at a second position, such as a test area. By comparing the at least two measured values with each other, as opposed to comparing a single measured value with a known control value, a measured parameter differential profile is obtained which is correlated to a hydration or moisture characteristic assessment. This assessment is independent of the effects of such variables as environmental conditions and subject variability during measurement, because the correlation is based upon the relative differences between the at least two values for each corresponding area on the skin.

In one embodiment, the present invention is directed to a method for measuring moisture content of skin. The method comprises the steps of: (a) providing a means for measuring skin moisture content; (b) using the means for measuring skin moisture content to obtain at least two measured moisture content values for the skin; (c) comparing the at least two measured moisture content values with each other to obtain a measured moisture content differential; and (d) correlating the measured moisture content differential to a skin moisture value for the skin.

In another embodiment, the present invention is directed to a method for measuring moisture content of scalp. The method comprises the steps of: (a) providing a moisture meter for measuring scalp moisture content; (b) using the meter to obtain at least a first measured moisture content value for the scalp at a position off the scalp; (c) using the meter to obtain at least a second measured moisture content value for the scalp at a position on the scalp; (d) comparing the first measured moisture content value with the second measured moisture content value to obtain a measured moisture content differential; and (e) correlating the measured moisture content differential to a scalp moisture value for the scalp.

In yet another embodiment, the present invention is directed to a method for measuring dandruff conditions on scalp of a consumer. The method comprises the steps of: (a) providing a moisture meter for measuring scalp moisture content; (b) using the meter to obtain at least two measured moisture content values from head of the consumer; (c) comparing the at least two measured moisture content values with each other to obtain a measured moisture content differential; and (d) correlating the measured moisture content differential to a dandruff condition indicator value for the consumer.

These and other features, aspects and advantages of the present invention will become evident to those skilled in the art from a reading of the present disclosure.

### BRIEF DESCRIPTION OF THE DRAWINGS

While the specification concludes with claims which particularly point out and distinctly claim the invention, it is believed the present invention will be better understood from the following description taken in conjunction with the accompanying drawings.

FIG. 1 is a flow diagram of the process of an embodiment of the present invention; and

FIG. 2 is a flow diagram of the process for measuring moisture content of the scalp of an embodiment of the present invention.

### DETAILED DESCRIPTION OF THE INVENTION

As used herein, the term “skin” refers to the membranous tissue forming the external covering of a mammalian body including, for example, the external covering of the face, neck, chest, back, arms, hands, legs and scalp.

As used herein, “moisture content” refers to the water present in the skin.

As used herein, “control area” refers to an area of the skin that is exposed to the environment and convenient for measurement.

As used herein, “test area” refers to the area of skin that a consumer wishes to have tested in order to select an appropriate skin treatment for use by consumer on that area.

According to the present invention, methods are described for measuring the moisture content of skin and methods of treating skin. The determination of moisture content in skin, including scalp, is used to quantify various physical and cosmetic characteristics of the skin. For example, a scalp with a low moisture content is unhealthy and may exhibit signs of dandruff, including flakes, dryness, tightness, itchiness and/or redness/irritation. Skin hydration is a function of its normal biological activity that results in continuous moisture flux from within the body to the environment. The improvement of skin barrier function results in greater skin hydration and less moisture loss. As a result, the physical and cosmetic characteristics of the skin may be improved, for example, with treatments that restore skin to normal conditions and improve its barrier function. Improvement of barrier function, in turn, also results in protecting the skin from environmental, physical, chemical, or biological insults and results in an overall improvement in skin health.

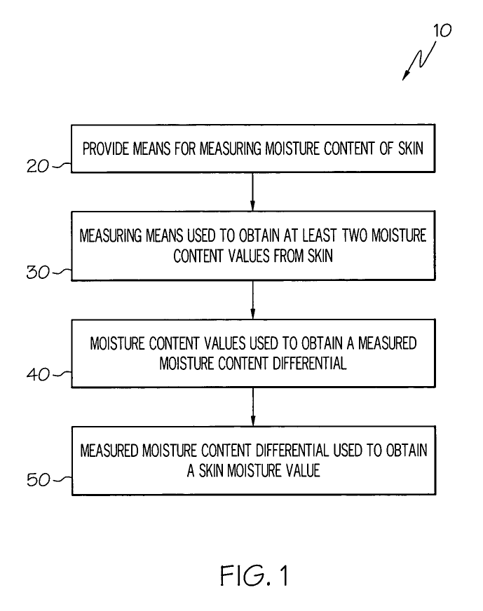

Referring now to FIG. 1, method for measuring moisture content of skin 10, contains a series of steps which are intended to provide information to a recipient or user for purposes of assessing the moisture level of skin. In a first step 20, a means for measuring skin moisture content is provided. Any suitable means for measuring skin moisture content may be used. For example, an electronic device comprising an impedance sensor, as described in U.S. Patent Application entitled “Method and Device for Indicating Moisture Content of Skin” filed on Sep. 2, 2005, Attorney Docket No. 10121P, can be used to measure the moisture content of skin. Suitable means for measuring moisture of skin also include radio frequency, infrared, nuclear magnetic resonance, mechanical vibration, skin deformation, iontophoresis, topology, friction, trans epidermal water loss (TEWL), optical and heat dissipation.

In a second step 30, the means for measuring skin moisture content is used to obtain at least two measured moisture content values from skin. In one embodiment, measurement of the moisture content of the skin is made for at least two areas on the skin, a control area that is in equilibrium with the environment and a test area. In one example, the control area may include the face, neck, ears, arms, hands, legs, feet, abdomen, back and groin, while the test area may include, for example, the face, neck, chest, back, arms, hands, feet, legs and scalp. However, several such measurements at various positions on the test area can be produced. In one example, the means for measuring skin moisture content is used to obtain one or more additional measured moisture content values on the test area. As each measurement is captured by the measurement means it is displayed or stored in a suitable manner. In one example, the measured moisture content values are displayed on an LED display. In another example, the measured moisture content values are stored in a programmable integrated circuit. In one embodiment, the measured values obtained for the control area and the test area have a frequency of less than about 5 MHz, in another embodiment, less than about 2.4 MHz, and in yet another embodiment, less than about 0.5 MHz.

In a third step 40, the at least two measured moisture content values are compared to each other in order to obtain a measured moisture content differential. For example, the skin moisture content value for the control area (m1) is compared to the skin moisture content value for the test area (m2), to obtain a measured moisture content differential (m2-m1) or (m1-m2). In one embodiment, the measurement means is electrically coupled through a cable to a processing system, such as a conventional PC or laptop computer. In another embodiment, the measurement means is electrically coupled to a programmable integrated circuit. In yet another embodiment, the measurement means is electrically coupled to a computer wirelessly. The processing system is operable to convert the measured moisture content values generated by the measurement means into a measured moisture content differential. In another embodiment, the measured moisture content differential may be determined manually using the formula (m2-m1) or (m1-m2).

If additional test area skin moisture values are obtained, all of these values are compared to one another and the measurement with the largest value, lowest value or mean value is then compared with the skin moisture content value for the control area in order to obtain a measured moisture content differential profile. In one embodiment, a measurement differential from about 0 MHz to about 0.05 MHz, represents a healthy skin condition (high moisture level); a- measurement differential from about 0.05 MHz to about 0.5 MHz represents a moderate skin condition (medium moisture level); and a measurement differential greater than about 0.5 MHz represents an unhealthy skin condition (low moisture level).

In another embodiment, the control area and the test area could be the same. For example, a measurement of the moisture content is taken at the control area. A period of time is then allowed to elapse during which the control area may be treated with a skin or scalp treatment. Thereafter, a second measurement is taken at the control area. The two measured moisture content values are then compared to each other in order to obtain a measured moisture content differential. In effect, the control area and the test area are the same for the second measurement.

In a fourth step 50, the moisture content differential is correlated to a skin moisture value. In one embodiment, the processing system described above is operable to convert the frequency differential into a skin moisture value as shown in Table I.

TABLE I

| Measured Signal | | |
|---|---|---|
| Difference between | | |
| Control Area and Test | | |
| Area (MHz) | Skin Moisture Value | Skin Condition |
| >=0.9 | 5 | D |
| 0.79-0.89 | 10 | D |
| 0.68-0.78 | 20 | D |
| 0.54-0.67 | 30 | D |
| 0.43-0.53 | 40 | D |
| 0.34-0.42 | 50 | M |
| 0.25-0.33 | 60 | M |
| 0.15-0.24 | 70 | M |
| 0.06-0.14 | 80 | M |
| 0.04-0.05 | 90 | H |
| <=0.03 | 95 | H |

In one embodiment, the skin moisture value is from about 0 to about 99, and in another embodiment, from about 5 to about 95. Such a value is based upon the relative differences between the measured values for the control area and the test area of the skin. As such, the skin serves as its own control. The skin moisture value may also be represented by such terms as “healthy” or “H ”, “moderate ” or “M ”, and “dry” or “D,” however, any words, pictures, or numbered grade scales which depict increasing or decreasing quantities of skin moisture levels may be used in the invention.

In another aspect, the invention relates, additionally, to using the skin moisture value to select at least one appropriate skin treatment. As used herein, an “appropriate skin treatment ” is a chemical composition or non-chemical treatment which provides or restores skin with the physical or cosmetic characteristics desired by a consumer. Exemplary skin treatment chemical compositions may include, without limitation, beauty care products, healthcare products, cosmetics, baby care products, feminine care products and pet care products, in the form of, lotions, creams, gels, tonics, after shave, sticks, sprays, ointments, pastes, powders, mousse, shampoos, conditioners, oils, colorants, and biomedical and dermatological treatments.

Nonlimiting examples of biomedical and dermatological treatments include prescription skin care treatments, laser treatment, chemical peel, dermabrasion, electrical stimulation, botox treatments, surgical treatments and exfoliating pads and cloths.

Exemplary non-chemical treatment may include, without limitation, hair shaving and hair removal.

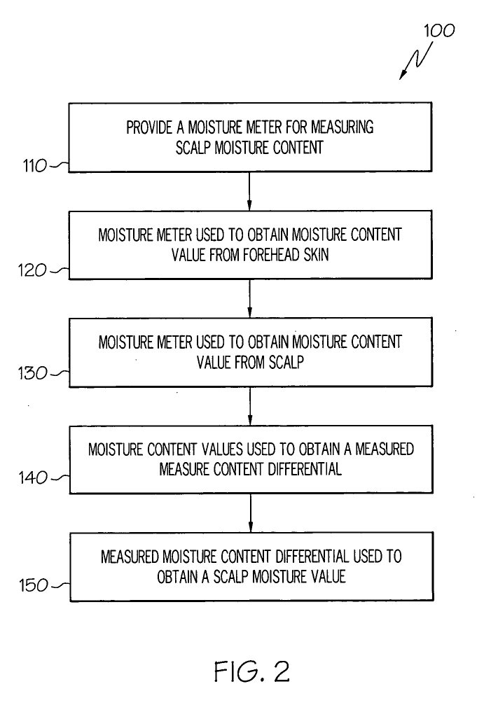

Referring now to FIG. 2, method for measuring moisture content of the scalp 100, contains a series of steps which are intended to provide information to a recipient or user for purposes of assessing the moisture level of the scalp. In a first step 110, a moisture meter for measuring scalp moisture content is provided. In one embodiment, the moisture meter may be an electronic device comprising an impedance sensor, as described above. In a second step 120, the moisture meter is used to obtain at least a first measured moisture content value at a position off the scalp. In one example, the off the scalp location may include, for example, forehead skin, cheek, chin, ear and arm.

In a third step 130, the moisture meter is used to obtain at least a second measured moisture content value at a position on the scalp. With respect to at least a second measured moisture value, several such measurements at various positions on the scalp can be produced. In one example, the moisture meter for measuring scalp moisture content is used to obtain one or more additional measured moisture content values at various locations on the scalp. As each measured moisture content value is captured in steps 120 and 130, the values are displayed in a suitable manner, for example, on an LED display on the moisture meter. In one embodiment, the moisture content values obtained for the position off the scalp location and the position on the scalp have a frequency of less than about 5 MHz, in another embodiment less than about 2.4 MHz, and in yet another embodiment less than about 0.5 MHz.

In a fourth step 140, the first measured moisture content value is compared with the second measured moisture content value to obtain a measured moisture content differential. For example, the scalp moisture content value for the position off the scalp (m1) is compared to the scalp moisture content value for the position on the scalp (m2), to obtain a measured moisture content differential (m2-m1) or (m1-m2). The moisture meter may be electrically coupled as described above. The processing system is operable to convert the measured moisture content values generated by the meter into a measured moisture content differential. In another embodiment, the measured moisture content differential may be determined manually using the formula (m2-m1) or (m1-m2).

If additional scalp moisture values are obtained at positions on the scalp, all of these values are compared to one another. The scalp moisture value with the largest value, or lowest value or mean value is then compared with the moisture content value for the position off the scalp. In one embodiment, a measurement differential from about 0 MHz to about 0.05 MHz, represents a healthy scalp condition (high moisture level); a measurement differential from about 0.05 MHz to about 0.5 MHz represents a moderate scalp condition (medium moisture level); and a measurement differential greater than about 0.5 MHz represents an unhealthy scalp condition (low moisture level).

In a fifth step 150, the moisture content differential is correlated to a scalp moisture value, in the same manner as described above with respect to the skin moisture value.

In another aspect of the invention, the method of this invention relates to a method of treating the scalp, including (a) measuring moisture content of said scalp according to the method described above; (b) using the correlated scalp moisture value to select at least one appropriate scalp treatment; and (c) applying the appropriate scalp treatment to the scalp. As used herein, an “appropriate scalp treatment” is a treatment which provides or restores the scalp with the physical or cosmetic characteristics desired by a consumer. Exemplary scalp treatment compositions may include, without limitation, shampoos, conditioners, oils, colorants, and styling compositions.

In another embodiment of the invention, the method of this invention relates to a method for measuring dandruff conditions on the scalp of a consumer, including (a) providing a moisture meter for measuring scalp moisture content; (b) using said meter to obtain at least two measured moisture content values from head of said consumer; (c) comparing said at least two measured moisture content values with each other to obtain a measured moisture content differential; and (d) correlating said measured moisture content differential to a dandruff condition indicator value for said consumer. In one example, dandruff conditions may include, for example, flakes, dryness, tightness, itchiness and redness/irritation. In one embodiment, the dandruff condition indicator value is from about 0 to about 99, and in another embodiment, from about 5 to about 95. The dandruff condition indicator value may also be represented by such terms as “healthy” or “H”, “moderate” or “M”, and “dry” or “D,” however, any words or a numbered grade scale which depict increasing or decreasing quantities of scalp moisture levels may be used in the invention.

### EXAMPLE

The following example is given solely for the purpose of illustration and is not to be construed as limitations of the present invention, as many variations of the invention are possible without departing from the spirit and scope of the invention.

### Example 1

Scalp Moisture Content and Dandruff

To determine a correlation between moisture content of scalp and dandruff, the scalp moisture content of consumers was measured using an electronic device comprising an impedance sensor as described above. The scalp moisture content for each consumer was determined as follows: One reading was taken from the forehead; and two readings were taken from the scalp. The measured moisture content values were then converted into a measured moisture content differential by the device. In addition, each consumer was assigned an Adherent Scalp Flaking Score (“ASFS”). The ASFS for each consumer was determined by having a qualified grader examine an octant of the consumer's scalp and then assigning a flake grade to that octant. Exemplary results are shown below Table II.

TABLE II

| | Measured Signal | | |
|---|---|---|---|
| Number of | Difference between | ASFS | |
| Consumers | m1 and m2 (KHz) | Octant Grade | Scalp Condition |
| 61 | 395.164 | 8-10 | Severe Flaking |
| 235 | 308.766 | 4-6 | Moderate Flaking |
| 169 | 230.527 | 0-2 | No Flaking |

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for measuring moisture content of skin, said method comprising the steps of:
a) providing a means for measuring skin moisture content;
b) using said means for measuring skin moisture content to obtain at least two measured moisture content values for said skin;
c) comparing said at least two measured moisture content values with each other to obtain a measured moisture content differential; and
d) correlating said measured moisture content differential to a skin moisture value for said skin.

2. The method according to claim 1, wherein said means for measuring skin moisture content is an electronic device.

3. The method according to claim 2, wherein said electronic device comprises an impedance sensor.

4. The method according to claim 1, wherein step (b) comprises obtaining a first measured moisture content value from a control area that is in equilibrium with environment and a second measured moisture content value on a test area.

5. The method according to claim 4, wherein said control area is selected from the group consisting of face, neck, ears, arms, hands, legs, feet, abdomen, back and groin.

6. The method according to claim 4, wherein said test area is selected from the group consisting of face, neck, chest, back, arms, hands, feet, legs and scalp.

7. The method according to claim 1, further comprising the step of using said skin moisture value to select at least one appropriate skin treatment.

8. The method according to claim 7, wherein said appropriate skin treatment is selected from the group consisting of chemical compositions and non-chemical treatments.

9. The method according to claim 8, wherein said chemical compositions are selected from the group consisting of lotions, creams, gels, tonics, after shave, sticks, sprays, ointments, pastes, powders, mousse, shampoos, conditioners, oils, colorants, and biomedical and dermatological treatments.

10. The method according to claim 8, wherein said non-chemical treatment is selected from the group consisting of hair shaving and hair removal.

11. The method according to claim 4, further comprising obtaining a third measured moisture content value for said skin at a second position on said test area.

12. A method for measuring moisture content of scalp, said method comprising the steps of:
a) providing a moisture meter for measuring scalp moisture content;
b) using said meter to obtain at least a first measured moisture content value for said scalp at a position off said scalp;
c) using said meter to obtain at least a second measured moisture content value for said scalp at a position on said scalp;
d) comparing said first measured moisture content value with said second measured moisture content value to obtain a measured moisture content differential; and
e) correlating said measured moisture content differential to a scalp moisture value for said scalp.

13. The method according to claim 12, wherein said moisture meter comprises an impedance sensor.

14. The method according to claim 12, wherein said moisture content values have a frequency of less than about 5 MHz.

15. The method according to claim 12, wherein said moisture content differential is from about 0 MHz to about 5 MHz.

16. The method according to claim 12, wherein said scalp moisture value is from about 0 to about 99.

17. A method of treating the scalp, said method comprising the steps of:
a) measuring moisture content of said scalp according to the method of claim 12;
b) using said correlated scalp moisture value to select at least one appropriate scalp treatment; and
c) applying said appropriate scalp treatment to said scalp.

18. The method according to claim 17, wherein said appropriate scalp treatment is selected from the group consisting of shampoos, conditioners, colorants, oils, and styling compositions.

19. The method according to claim 12, wherein said position off said scalp is selected from the group consisting of forehead skin, cheek, chin, ear and arm.

20. A method for measuring dandruff conditions on scalp of a consumer, said method comprising the steps of:
a) providing a moisture meter for measuring scalp moisture content;
b) using said meter to obtain at least two measured moisture content values from head of said consumer;
c) comparing said at least two measured moisture content values with each other to obtain a measured moisture content differential; and
d) correlating said measured moisture content differential to a dandruff condition indicator value for said consumer.
